(19) United States
(12) Patent Application Publication
(10) Pub. No.: US 2013/0309217 A1
(43) Pub. Date: Nov. 21, 2013

(54) COSMETIC USE

(71) Applicant: Alfred Schmidt, Nambsheim (FR)

(72) Inventor: Alfred Schmidt, Nambsheim (FR)

(73) Assignee: LUCOLAS-M.D. LTD., Birmingham (UK)

(21) Appl. No.: 13/982,539

(22) PCT Filed: Jan. 30, 2012
(86) PCT No.: PCT/EP12/51421

§ 371 Date: Jul. 30, 2013

Related U.S. Application Data
(60) Provisional application No. 61/444,795, filed on Feb. 21, 2011.

(30) Foreign Application Priority Data
Jan. 31, 2011 (DE) 10 2011 003 408.0

(51) Int. Cl. A61K 8/73; A61Q 19/00; A61Q 19/08; A61K 8/49; A61K 8/63

(52) U.S. Cl. CPC A61K 8/735; A61K 8/4986; A61K 8/63; A61Q 19/08; A61Q 19/00
USPC 424/94.1; 514/440

(57) ABSTRACT

The invention relates to the use of an agent and a composition, respectively, comprising the following active ingredients:
a) an aromatase inhibitor and/or a 5-α-reductase inhibitor,
b) an antioxidant, and
c) hyaluronic acid
   for cosmetic purposes.

The invention relates to the cosmetic use of a composition for improving the general condition and appearance of the skin.

Depending on the respective cultural environment or the respective epoch, the ideals of beauty are subject to certain transformations. Nevertheless, also today a flawless appearance is of great importance for the majority of the population. In this respect, the condition and the appearance of the skin play a decisive role.

The skin represents a very versatile organ, having a series of essential functions in the human or animal organism, respectively. For instance, on the other hand, the skin provides a barrier, which delimits the body externally and protects it from harmful environmental influences or allows for the exchange with the environment, respectively. On the other hand, the skin has important metabolic functions and is involved, for example, in a significant manner in the defense of pathogens, but also in allergic reactions.

As a consequence of e.g. nicotine and/or alcohol abuse and of the permanent exposition of the skin to influences from the environment such as, for example, UV-radiation, the so-called exogenic skin aging occurs. Moreover, endogenous factors such as the genetic predisposition, additionally cause an aging effect.

One consequence of skin aging is the development of wrinkles due to drying and loss of elasticity in the epidermis. This is accompanied by impaired wound healing and an overall thinner epidermal layer. This causes a stronger visibility of modified blood vessels, in particular in the case of spider veins. A further condition significantly compromising the appearance of the skin is cellulite. Cellulite is not a condition caused by a disease but rather an aesthetic problem, which occurs mostly in women. Therein, the deposition of fat in the subcutis is increased leading, in cases of connective tissue-weakness, to irregular dents on the skin, the so-called orange skin. Also the so-called stretch marks represent an impairment of life quality for many people. Stretch marks are formed by overstretching of the connective tissue in the subcutis, e.g. due to strong weight increase. The overstretching of the connective tissue first leads to blue-reddish stripes; due to the scarring of these tissue ruptures they appear as bright stripes later on, which are distinct from the surrounding skin to a varying degree depending on the pigmentation of the affected skin area.

The cosmetic industry offers various options aiming at counteracting signs of aging in general. The success of the respective products and methods in the long term, however, does often not come up to the expectations of the user. For instance, mainly two methods are used in order to remove spider veins, both of which are connected to side-effects and in both of which frequent relapses occur. Sclerotherapy is an invasive method, in which, besides pain, side-effects such as hematomas and venous thromboses can occur. In laser treatment, which frequently requires multiple sessions, allergic skin reactions and pain can occur as side effects. Recurrence rates are relatively high in both methods.

There is therefore a need for cosmetic uses, which counteract the phenomena mentioned above and signs of skin aging in general.

It is an object of the present invention to provide a cosmetic use of a composition for improving the appearance of the skin. In particular, the visible consequences of cellulite, stretch marks, spider veins and signs of aging in general shall be reduced or avoided.

This objective is solved by the present invention as defined in claim 1. Embodiments are defined in the respective sub-claims.

The present invention relates to the use of an agent or a substance, in particular of a composition, which comprises the following active ingredients:
a) an aromatase inhibitor and/or a 5-α-reductase inhibitor,
b) an antioxidant and
c) hyaluronic acid
   for cosmetic purposes.

In the use according to the invention the single components a), b) and/or c) can each be used independently from each other together, i.e. in a common composition, or alternatively separately in separated forms, in the latter case however, in such a manner that at least they are applied during the same period of time. The use in a common composition is preferred.

The skin condition is substantially influenced by the microcirculation in the skin. The latter is obstructed, for example, by the size increase and multiplication of fat cells, respectively. The increase of fat cells or the fat contents in the cells, respectively, reduces the skin's capacity to bind water and leads to the formation of wrinkles.

In the context of the present invention, it has surprisingly been found that the use of the composition according to the invention, which besides an aromatase inhibitor and/or a 5-α-reductase inhibitor can also comprise an antioxidant and hyaluronic acid, substantially improves the appearance of the skin. The use according to the invention does not only lead to the reduction of wrinkles, but also to an improvement of cellulite, stretch marks, spider veins as well as of the general appearance of the skin surface. In particular, signs of aging (e.g. age spots, eye wrinkles, general wrinkles in the face, etc.), aging of the decolleté or signs of UV-aging (photo aging) are improved by the use according to the invention.

Without wishing to limit the scope of the present invention by a theory, it is assumed that the effect according to the invention is based on the improvement of the microcirculation in the skin. This does not only lead to increased water-binding in the skin, but also to a strengthening of the collagen fibers and thus to a smooth skin surface. Furthermore, the microcirculation improved by the use according to the invention, apparently also enhances metabolic processes in the skin. It is presumed that harmful metabolic products, which in the case of insufficient microcirculation accumulate in the skin, are metabolized and/or removed via the lymph. Thereby, in addition to the hormonal balance, also the metabolic equilibrium of the skin is re-established and the connective tissue is strengthened. It is assumed that simultaneously the effect of harmful radicals is reduced or totally avoided by the effect of the anti-oxidant providing better protection of the skin against new damages. By this approach, which aims at eliminating the central causes of the majority of skin damages, the most diverse phenomena can thus be treated that have a negative impact on the skin's appearance. The skin's general condition is improved by an improved supply of the skin and an improved protection of the skin against damages caused by radicals and dehydration. It is further assumed that the unexpected improvement of the skin's appearance is caused by a synergistic cooperation of the single components of the composition according to the invention. The conversion of testosterone to estrogen is locally reduced or blocked by the effect of the aromatase inhibitor and/or the 5-α-reductase inhibitor, respectively. This leads, amongst other effects, to a reduced increase of fat cells and/or to a decrease of the volume/fat content of the fat cells, respectively. At the same time, anabolic processes are enhanced, which contribute to the tightening of connective tissue and to wound healing. In particular, the simultaneous use of hyaluronic acid is important since it additionally enhances the effects of the aromatase inhibitor and of the 5-α-reductase inhibitor, respectively, by improving the absorption of these active ingredients and that of the antioxidant and by retaining the active ingredients in the skin.

As mentioned above, the antioxidant protects against free radicals and thus reduces or blocks a new damage of the skin. Apparently, the effects of the single active ingredients amplify each other therein in a disproportionate manner, which leads to the unexpected improvement of the skin's general condition.

The invention shall be illustrated in detail by the following description of preferred embodiments, without, however, limiting the general concept according to the invention thereto.

The present invention relates to the use of an agent and a composition, respectively, which, besides an aromatase inhibitor and/or a 5-α-reductase inhibitor, also contains an antioxidant and hyaluronic acid, for cosmetic purposes.

In the meaning of the present invention, the use of an antioxidant in combination with an aromatase inhibitor and/or a 5-α-reductase inhibitor and the further component hyaluronic acid, counteracts the aging process of the skin and improves—mainly due to the combination with hyaluronic acid—the microcirculation in the skin.

Aromatase inhibitors in the meaning of the present invention are all substances, which—independently from their structure—are characterized by the common feature that they effectively inhibit or even inactivate aromatase (review article: Santen et al., Endocrine Reviews 2009; 30:343-375). The capacity of substance to inhibit and inactivate, respectively, aromatase, can be determined by methods known to the skilled person. A radiometric assay allows for measurement of aromatase activity in just one step via determination of tritium-release from a tritium-labeled substrate (Thompson and Siiteri, Journal of Biological Chemistry 1974; 249:5364-5372). The group of aromatase inhibitors is structurally heterogenous and comprises steroidal as well as non-steroidal compounds, wherein representatives of both groups are relevant for the medication in the meaning of the invention. As non-steroidal aromatase inhibitors, e.g. Anastrazol, Letrozol and Vorozol can be used. Preferred steroidal aromatase inhibitors (aromatase-inactivators) are 4-hydroxyandrostenedione, Exemestane, 4-acetoxyandrostenedione, 5-α-androst-3-ene-17-one and 3-α, 4-α-epoxy-5-α-androstane-17-one.

5-α-reductase inhibitors in the meaning of the present invention are all substances, which reduce or completely inhibit the activity of 5-α-reductase type I and/or type II. Examples of 5-α-reductase inhibitors, which can be used according to the invention, are, amongst others, the extract from the fruits of saw palmetto (Serenoa repens, syn. Sabal serrulata), roots of stinging nettle (Urtica dioica), bark extract of African plum (Pygeum africanium), extract from pumpkin seeds (Cucurbita pepo seed) or Finasteride.

According to the present invention, the aromatase is preferably of an effectiveness, which is characterized by an average inhibitory concentration IC(50) of from 0.2 nM to 500 nM.

The 5-α-reductase inhibitor in the meaning of the invention is preferably characterized by an average inhibitory concentration IC(50) from 5 nM to 500 nM.

An antioxidant according to the present invention is a “radical scavenger”, which captures free radicals or terminates their detrimental influence on the cell. The antioxidant's substance should differ from the aromatase inhibitor. While the antioxidants' mechanism of action is of the same direction and consistent in the meaning of the invention, the group of antioxidants is structurally very heterogenous. Suitable substances in the meaning of the invention are selected according to their capacity to prevent the oxidation of other molecules. The skilled person is capable of identifying an antioxidant by established and published methods. The processes are known to a skilled person and shall not be explained here in detail. Thus, e.g. the amount of free radicals can be measured by EPR (electric paramagnetic resonance; Lo Scalzo, EJEAFChe 2010; 9:1360-1371). Therefore, substances such as 5,5 -dimethyl-1-pyrrolin-N-oxide (DMPO) or 1,1-diphenyl-2-picrylhydrazyl (DPPH) are used, which have a high affinity to free radicals and together with these form stable compounds that can be measured spectrometrically. Processes are also used, in which the substance to be measured is purified chromatographically (e.g. via HPLC; Yamaguchi et al., Bioscience, Biotechnology, and Biochemistry 1998; 62:1201-1204). In the meaning of the present invention, antioxidative substances are used. The used antioxidants can be substances of various chemical classes and different origin. These can also be antioxidant substances, which occur in the organism, which amount and availability, respectively, are effectively increased, however, by the additional administration in the context of the use according to the invention, and/or which are eventually provided just at the desired target site by suitable application. Non-enzymatic antioxidants comprise, in particular, flavonoids (e.g. oligomeric proanthocyanidines (OPC), anthocyanes or polyphenoles such as quercetin or catechin); vitamins (e.g. vitamin C, vitamin E); carotenoids (e.g. β-carotin, lycopen, lutein); minerals (e.g. copper, manganese, zinc, selenium); hormones (e.g. melatonin); steroids (e.g. cortisol); ubiquinones; N-acetylcysteine; α-lipoic acid; and an extract of green tea containing an antioxidative effective composition of polyphenols, optionally also amino acids, mineral nutrients (trace elements) and polysaccharides, in particular which contains the specific highly antioxidative acting polyphenols epicatechin and epigallocatechin (e.g. OM24®, obtainable from Omnimedica, Switzerland); and glutathione. Some enzymes fulfill the function of antioxidants and are called enzymatic anti-oxidants such as e.g. glutathione peroxidase, superoxide dismutase and katalase.

In comparison with possibly only endogenously occurring, or if so only randomly or for other purposes added antioxidants, according to the invention it can be ensured by adjusting appropriate amounts or by topical application that a desired cosmetic effect is achieved.

In a particular embodiment, the antioxidant, which is used in the cosmetic composition, is α-lipoic acid (1,2-dithiolan-3-pentanoic acid) or green tea extract containing polyphenols, in particular OM24®. α-lipoic acid is active in the aqueous as well as in the lipid phases of the cells. The substance is excellently resorbed via the skin. This is particularly the case in combination with hyaluronic acid. This allows for different possibilities of administration. α-lipoic acid is readily converted into dihydro-lipoic acid in the organism. Dihydro-lipoic acid regenerates other further antioxidants such as vitamin C and vitamin E, which can lead to further amplified effects in the administration of α-lipoic acid. α-lipoic acid furthermore induces the synthesis of glutathione in the tissue.

Moreover, α-lipoic acid regenerates glutathione from glutathione disulfide.

In the topical use of the composition on the skin the active ingredients are applied to the skin area to be treated. The amount of active ingredients therein is preferably selected in such a manner that no plasma levels but only locally effective concentrations are reached. As a consequence, undesired systemic effects can be avoided. In this manner, undesired side-effects can be avoided even in the use over a longer period.

Alternatively, other application forms can be used, such as e.g. spraying the active ingredients onto the skin areas to be treated.

Therein, the active ingredients can be applied separately from each other or in a common composition, as long as it is ensured that the active ingredients reach the target tissue at the same time or that the time intervals at least overlap, in which the single active ingredients are present in the target tissue as active substances.

In a preferred embodiment, the use according to the invention comprises α-lipoic acid and hyaluronic acid in combination with an aromatase inhibitor and/or a 5-α-reductase inhibitor. In a particularly preferred embodiment, the use according to the invention comprises α-lipoic acid, hyaluronic acid and 4-acetoxyandrostenedione. The use of green tea extract containing polyphenols, in particular OM24® as antioxidant instead of or in addition to the use of α-lipoic acid is also preferred.

Other further excipients, which are commonly used in cosmetic compositions (creams, ointments, gels, foams, tinctures, lotions, etc.), can be combined with the active ingredients mentioned above. In particular, the composition according to the invention can comprise excipients, which are commonly used in topical application forms. Likewise, respective carriers and excipients can be used for the provision as a spray, that are known to the skilled person.

In the meaning of the invention, the preferred concentrations of the aromatase inhibitor in the composition are in a range from 0.25 weight-% to 1.5 weight-% (e.g. in cellulite 0.6 weight-%).

According to the invention, the concentration of the 5-α-reductase inhibitor is in the range from 0.5 weight-% to 5 weight-%.

The antioxidant is preferably present in a concentration of from 0.2 weight-% to 2.5 weight-% in the composition.

The composition is typically applied to the respective skin areas 1-2 times per day. Therein, normally 1-5 g of, for example, a cream or 2-5 ml of, for example, a spray are used for each application.

The present application is further illustrated by the following examples, without being limited by these.

The following Examples 1 and 2 illustrate the effectiveness of the use according to the invention in the case of spider veins.

The following composition has been used therein:
1.0% acetoxyandrostenedione
0.5% α-lipoic acid
0.5% hyaluronic acid
   as active ingredients

in DAC basic cream:
4.0 g glycerolmonostearate
6.0 g cetyl alcohol
7.5 g medium chain triglycerides (neutral oil, miglyol)
25.5 g white Vaseline
7.0 g Macrogol-20-glycerolmonostearate
10.0 g propylenglycol
40.0 g purified water.

### EXAMPLE 1

S.H.C., 48 years, female: spider veins at the inner parts of the thighs at knee level on both sides as well as a strong circular accumulation (diameter about 1.5 cm) on the backside of the left thigh slightly above the knee.

After twice-daily treatment for four weeks with the cream according to the invention: the circular accumulation on the backside is already dissolved, only spider veins are visible, which are elongated but continue to become lighter. The treatment is continued as completely without side-effects.

### EXAMPLE 2

E.C., 51 years, female: spider veins at the outer parts of both thighs and at the calves.

After twice-daily treatment with the cream according to the invention over a period of three months: almost no visible spider veins remaining.

The treatment is continued with application once daily as completely without side-effects.

The following Example 3 shows the results of female trial subjects with cellulite. The following cream composition has been used therein once per day (about 1.5 g per afflicted body side; %-indications in weight-%):
0.6% acetoxyandrostenedione
0.5% α-lipoic acid
0.2% hyaluronic acid
   as active ingredients

in DAC basic cream:
4.0 g glycerolmonostearate
6.0 g cetyl alcohol
7.5 g medium chain triglycerides (neutral oil, miglyol)
25.5 g white Vaseline
7.0 g Macrogol-20-glycerolmonostearate
10.0 g propyleneglycol
40.0 g purified water.

### EXAMPLE 3

### Observational Study With 50 Female Trial Subjects

The subjects were treated in 5 Swiss health studios. Only subjects having a cellulite expression of score points 2 and 3 of the cellulite evaluation score according to Nürnberger & Müller (Nürnberger F., Müller G.: So-called Cellulite: an invented disease. J. Dermatol. Surg. Oncol. 1978, 4: 221-9) were included.

Nürnberger Score: 0=no orange skin (cellulite)
1=mild expression
2=moderate expression
3=strong expression

The study proceeded for 12 weeks. Observation time points 0: prior to treatment/beginning of treatment; further time points for examination after week 4, 8 and 12.

The subjects had an average age of 36 years (19-57 years), were healthy and of normal weight or slightly overweight. Therapy results (after 12 weeks; evaluated according to Nürnberger score):

At the beginning of the therapy: 29 subjects; score 2
21 subjects; score 3

All test persons underwent the 12 week observation period; results after 12 weeks of application:

Out of 29 subjects with moderate expression at the beginning, 20 subjects had no more cellulite (score 0), 9 test persons score 1, mild expression. Out of the 21 score 3 subjects, 8 subjects had score 2 after 12 weeks; 11 score 1; 2 score 0. None of the subjects complained about undesired effects; the cream was perceived as very pleasant and effective. For the maintenance or the further improvement of the results, respectively, the subjects continue the application, namely with the identical cream composition.

1-10. (canceled)

11. A method for improving the general condition and appearance of skin, the method comprising treating a patient for cosmetic purposes with a composition comprising the following active ingredients:
an aromatase inhibitor and/or a 5-α-reductase inhibitor,
an antioxidant, and
hyaluronic acid.

12. The method according to claim 11, wherein the aromatase inhibitor is selected from the group consisting of 4-hydroxyandrostenedione, Exemestane, 4-acetoxyandrostenedione, 5-α-androst-3-ene-17-one and 3-α,4-α-epoxy-5-α-androstane-17-one.

13. The method according to claim 12, wherein the aromatase inhibitor has a mean inhibitory concentration IC(50) in the range from 0.2 nM to 500 nM.

14. The method according to claim 11, wherein the antioxidant is selected from the group consisting of flavonoids; vitamins; carotenoids; minerals; hormones; steroids; ubiquinones; N-acetylcysteine; α-lipoic acid; green tea extract containing polyphenols; glutathione; glutathione peroxidase; superoxide dismutase and katalase.

15. The method according to claim 11, wherein the antioxidant is a composition of polyphenols, amino acids, mineral nutrients (trace elements) and polysaccharides.

16. The method according to claim 11, wherein the 5-α-reductase inhibitor inhibits the activity of 5-α-reductase type I and/or type II.

17. The method according to claim 11, wherein the 5-α-reductase inhibitor has a mean inhibitory concentration IC(50) is in the range from 5 nM to 500 nM.

18. The method according to claim 11, wherein the 5-α-reductase inhibitor is selected from the group consisting of extract from the fruits of saw palmetto (Serona repens, syn. Sabal serrulata), roots of stinging nettle (Urtica dioica), bark extract of African plum (Pygeum africanium), extract from pumpkin seed (Cucurbita pepo seed) or Finasteride.

19. The method according to claim 11, wherein the composition is administered topically to the skin.

20. The method according to claim 11, wherein the composition comprises (i) 4-acetoxyandrostenedione, (ii) saw palmetto extract, (iii) α-lipoic acid and/or green tea extract containing polyphenols, and (iv) hyaluronic acid.

21. The method according to claim 11, wherein the composition comprises (i) 4-acetoxyandrostenedione, (ii) saw palmetto extract, (iii) α-lipoic acid and/or an antioxident composition of polyphenols, amino acids, mineral nutrients (trace elements) and polysaccharides, and (iv) hyaluronic acid.

22. The method according to claim 11, wherein the method comprises treating a patient for cosmetic purposes related to a condition selected from the group consisting of cellulite, stretch marks, signs of skin aging, wrinkles and spider veins.
